(19) United States
(12) Patent Application Publication
(10) Pub. No.: US 2013/0218208 A1
(43) Pub. Date: Aug. 22, 2013

(54) ROD-RECEIVING SPINAL FUSION ATTACHMENT ELEMENTS

(71) Applicant: Joseph Khoury, Birmingham, AL (US)

(72) Inventor: Joseph Khoury, Birmingham, AL (US)

(73) Assignee: The UAB Research Foundation, Birmingham, AL (US)

(21) Appl. No.: 13/768,723

(22) Filed: Feb. 15, 2013

Related U.S. Application Data
(60) Provisional application No. 61/599,540, filed on Feb. 16, 2012.

(51) Int. Cl. A61B 17/70

(52) U.S. Cl. CPC A61B 17/7001
USPC 606/278

(57) ABSTRACT

In one embodiment, an implantable spinal fusion attachment element includes a rod attachment head having a passage that is adapted to receive a spinal alignment rod and means for securely attaching the attachment element to a vertebra without penetrating the pedicle of the vertebra.

### CROSS-REFERENCE TO RELATED APPLICATION(S)

This application claims priority to co-pending U.S. Provisional Application Ser. No. 61/599,540, filed Feb. 16, 2012, which is hereby incorporated by reference herein in its entirety.

### BACKGROUND

Spinal fusion is commonly performed in the United States to correct scoliosis. During a spinal fusion procedure, the spine is exposed from the back by moving the muscles aside and spinal fusion hardware is implanted in order to correct the undesired spinal curvature. Bone graft material is then inserted between the vertebrae to cause the vertebrae to fuse together.

Spinal fusion implants have evolved over time and now provide great power of correction. In most cases, screws are passed through the pedicles of the vertebrae from back to front, and rods are then passed through openings formed in the heads of the screws. The rods maintain the desired orientation of the spine and enable the vertebrae to fuse in that orientation.

The pedicles are bordered on one side by the spinal canal (which contains the spinal cord) and by large blood vessels on the other side. When the scoliosis is severe, the pedicles are often malformed, very narrow, or even absent on the concave side of the curve. In such situations, screws cannot be passed through the pedicles. The surgeon must therefore either skip over the vertebrae or use an alternative form of fixation. When skipping over the vertebrae, less correction will be achieved.

Alternative forms of fixation include wires or ribbons that loop underneath the lamina and hooks. The disadvantage of wires or ribbons is that a pathway must be made between the underside of the lamina and the spinal cord. Forming such a pathway is time consuming and can cause significant bleeding. Also, the corrective power is limited. The disadvantage of hooks is that hooks can slip out during placement of the rod or the corrective maneuver.

From the above discussion, it can be appreciated that it would be desirable to have alternative means for fusing the spine.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is an exploded perspective view of a first embodiment of a spinal fusion attachment element.

FIG. 2 is a perspective view of the attachment element of FIG. 1 shown assembled.

FIG. 3 is an exploded perspective view of a second embodiment of a spinal fusion attachment element.

FIG. 4 is a perspective view of the attachment element of FIG. 3 shown assembled.

FIG. 5 is a superior view of a vertebra with the spinal fusion attachment element of in FIG. 1 attached to the vertebral body.

FIG. 6 is a posterior view of the vertebra and attachment element shown in FIG. 5.

FIG. 7 is a posterior view of vertebrae with two spinal fusion attachment elements similar to that shown in FIG. 1 attached to the vertebrae and an alignment rod associated with the attachment elements.

FIG. 8 is a perspective view of a third embodiment of a spinal fusion attachment element.

FIG. 9 is a side view of the attachment element of FIG. 8.

FIG. 10 is a side view of a fourth embodiment of a spinal fusion attachment element.

FIG. 11 is a superior view of a vertebra with the attachment element of FIG. 8 passed through a transverse process of the vertebra.

FIG. 12 is a posterior view of the vertebrae and attachment element shown in FIG. 11.

FIG. 13 is an exploded perspective view of a fourth embodiment of a spinal fusion attachment element.

### DETAILED DESCRIPTION

As described above, it would be desirable to have an effective alternative means for fusing the spine that does not require the use of pedicle screws. Disclosed herein are rod-receiving spinal fusion attachment elements that are adapted to attach to the vertebrae and receive an alignment rod used to correct lateral curvature of the spine. In some embodiments, the attachment elements comprise claws that grip the top surface and the lateral surface of the vertebral body. In other embodiments, the elements comprise studs that are passed through the transverse processes of the vertebrae. In both cases, the attachment elements comprise a head that is adapted to receive the rod.

In the following disclosure, various embodiments are described. It is to be understood that those embodiments are example implementations of the disclosed inventions and that alternative embodiments are possible. All such embodiments are intended to fall within the scope of this disclosure.

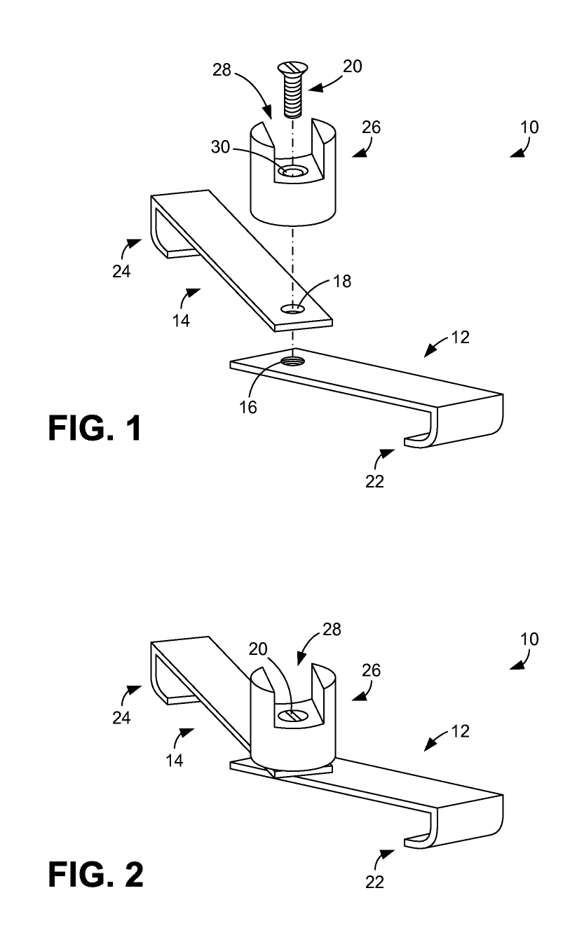

FIGS. 1 and 2 illustrate a first embodiment of a rod-receiving spinal fusion attachment element 10 that can, for example, be attached to a thoracic vertebra. The attachment element 10 is intended to provide a stable alternative to a pedicle screw when placement of the screw is difficult, dangerous, or impossible. As is shown in the figures, the attachment element 10 comprises two separate pieces, a first or lower arm 12 that is designed to grip the vertebral body below the transverse process and a second or upper arm 14 that is designed to grip the top of the vertebral body just medial of the facet joint. Each arm 12, 14 is formed from an elongated, planar, rectangular member, such as a stainless steel or titanium bar. At a first or proximal end of each arm 12, 14 is an opening 16, 18 that is adapted to receive the shaft of a fastener 20 that is used to secure the arms together. At a second or distal end of each arm 12, 14 is a gripping element 22, 24 that is used to grip the vertebra. In some embodiments, the gripping elements 22, 24 comprise hooks that extend downward from a plane in which the remainder of the arm 12, 14 lies and inward toward the proximal end of the arm.

As is also shown in FIGS. 1 and 2, the attachment element 10 further comprises a rod attachment head 26 that includes a passage 28 that is adapted to receive an alignment rod that is used to maintain a desired orientation of the spine. In some embodiments, the head 26 is a multi-axial tulip head that can pivot to accommodate the orientation of the rod. Formed through the bottom of the passage 28 is an opening 30 through which the shaft of the fastener 20 can pass. Therefore, as depicted in FIG. 1, the fastener 20 can pass through the head 26, the upper arm 14, and into the lower arm 12. In some embodiments, the shaft of the fastener 20 and the opening 16 of the lower arm 12 are both threaded so that once the fastener is threaded into the opening of the lower arm, the head 26, upper arm 14, and lower arm are all fixedly secured together in the orientation in which they are positioned when the tightening is performed (see FIG. 2). In other embodiments, a further component, such as a nut (not shown), can be associated with the lower arm 12 and used to assemble the attachment element 10.

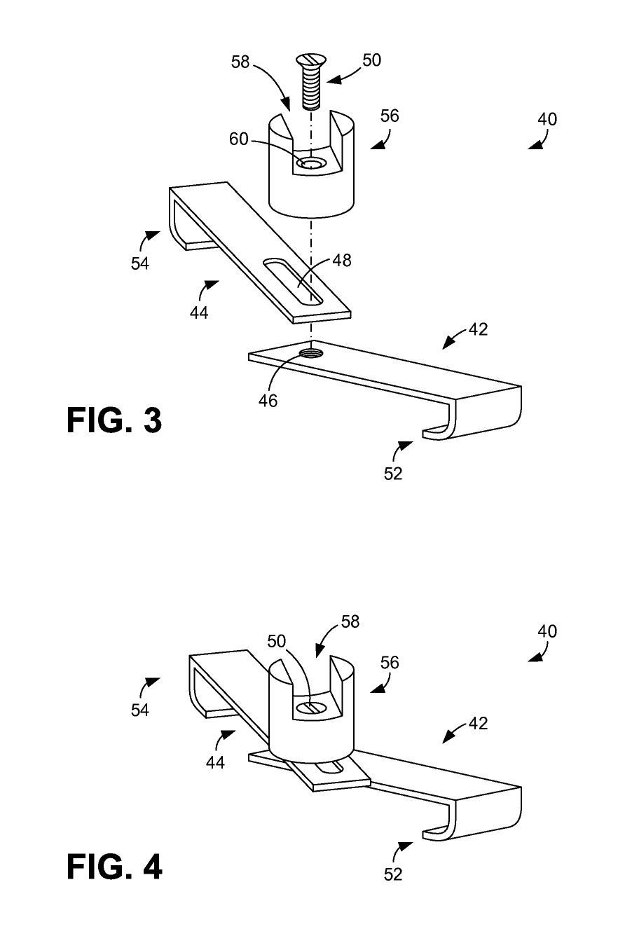

FIGS. 3 and 4 illustrate a second embodiment of a rod-receiving spinal fusion element 40, which can also attach to a thoracic vertebra. The attachment element 40 is similar in many ways to the attachment element 10 shown in FIGS. 1 and 2. Accordingly, the attachment element 40 comprises a first or lower arm 42, a second or upper arm 44, each arm comprising an opening 46, 48 at one end and a gripping element 52, 54 at the other end. In addition, the attachment element 40 comprises a rod attachment head 56 that includes a passage 58 that is adapted to receive an alignment rod that is used to maintain a desired orientation of the spine. An opening 60 is provided at the bottom of the passage 58. In the embodiment of FIGS. 3 and 4, however, the opening 48 provided in the upper arm 44 is formed as an elongated slot that extends along a length direction of the upper arm, so that the upper arm can be moved relative to the lower arm 42 to enable adjustment of the spacing between the gripping elements 52, 54 and accommodate the size of the patient's vertebra.

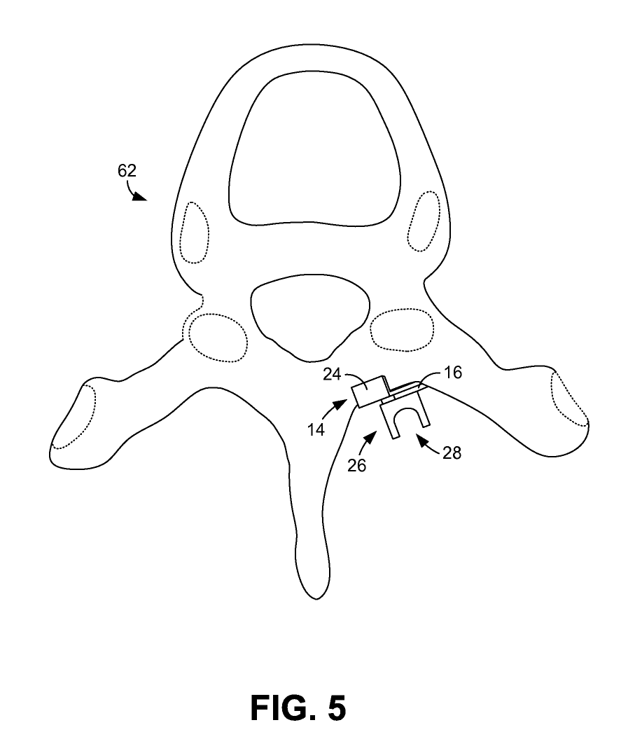

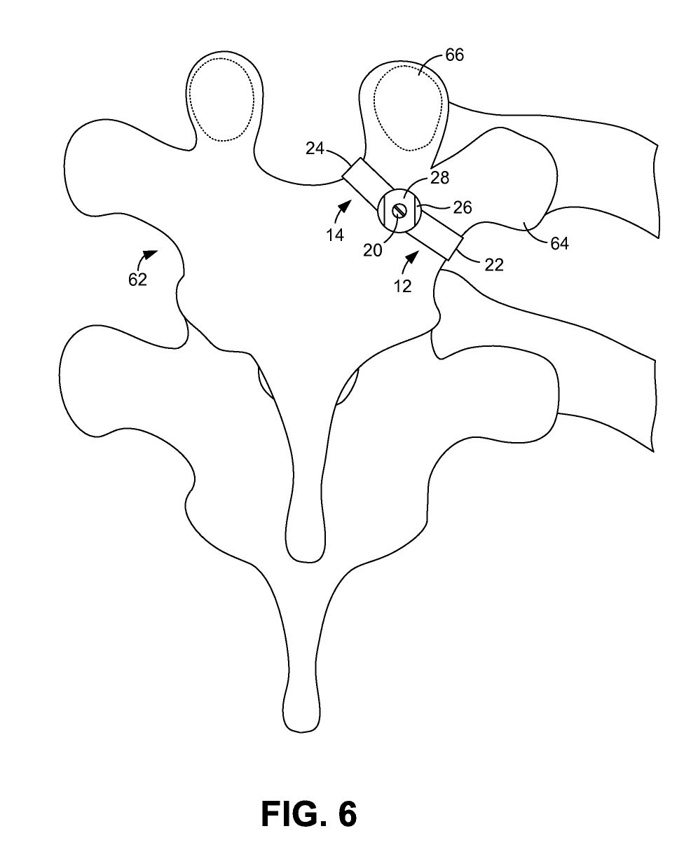

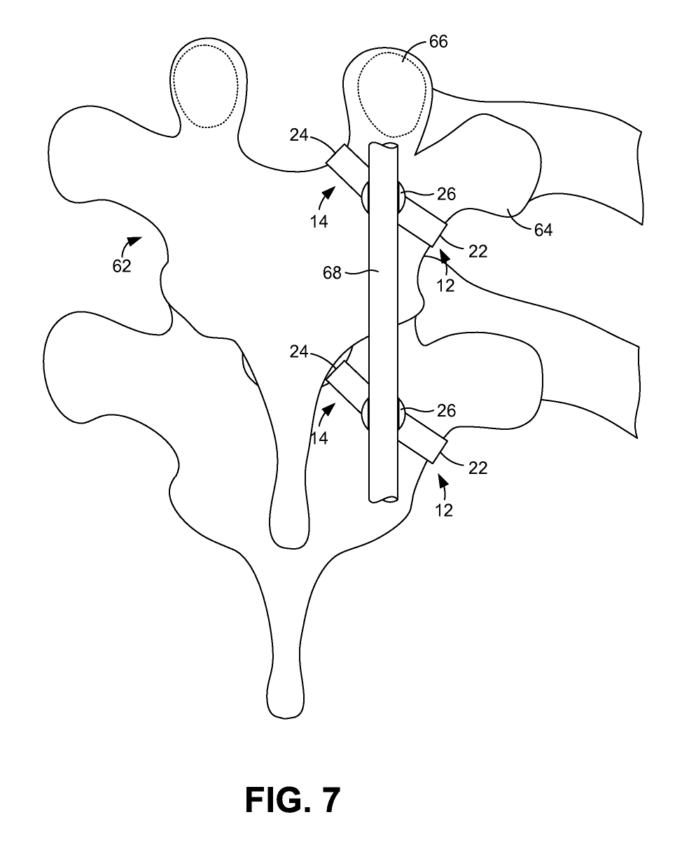

FIGS. 5 and 6 illustrate the attachment element 10 of FIGS. 1 and 2 attached to a vertebra 62. As is shown in these figures, the gripping element 22 of the lower arm 14 grips the vertebral body below a transverse process 64 and the gripping element 24 of the upper arm 14 grips the vertebral body just medial of a facet joint 66. Once the attachment element 10 has been attached in this manner, an alignment rod can be attached to the head 26 of the element. When multiple attachment elements 10 have been attached to multiple vertebrae, the rod can be attached to each of the elements. Such an arrangement is illustrated in FIG. 7, which shows an alignment rod 68 positioned within the heads of multiple attachment elements 10.

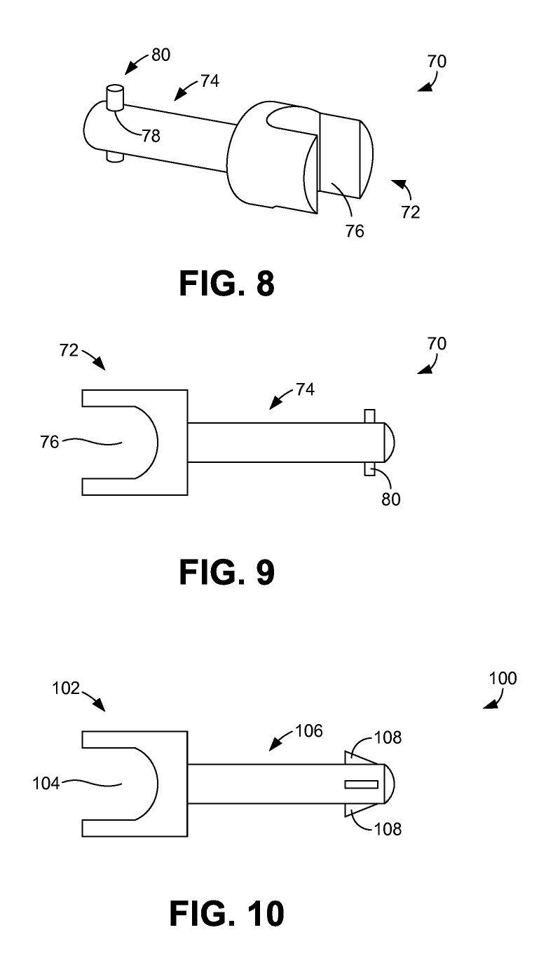

FIGS. 8 and 9 illustrate a third embodiment of a rod-receiving spinal fusion attachment element 70 configured as a stud that, for example, can be attached to a thoracic vertebra. In such a case, the attachment element 70 can be referred to as a thoracic stud. Like the attachment elements 10, 40, the attachment element 70 is intended to provide a stable alternative to a pedicle screw when placement of the screw is difficult, dangerous, or impossible.

As shown in the figures, the attachment element 70 comprises a rod attachment head 72 and a shaft 74 that extends outward from the head. Like the heads 26 and 56, the head 72 comprises a passage 76 that is adapted to receive a rod that is used to maintain a desired orientation of the spine. The shaft 74 is generally cylindrical and comprises a transverse opening 78 at its distal end that is adapted to receive a retainer element 80 that can, for example, comprise a small rod such as a small metal shaft or a thick piece of suture.

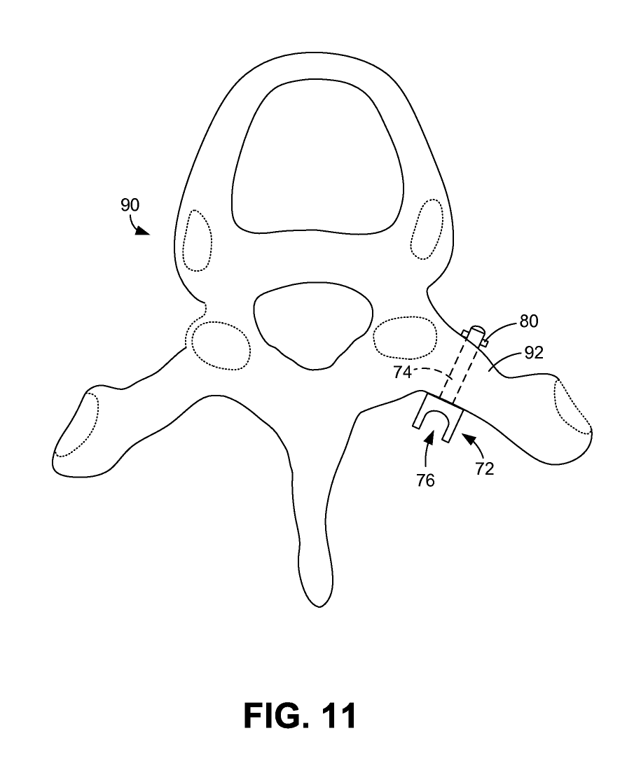

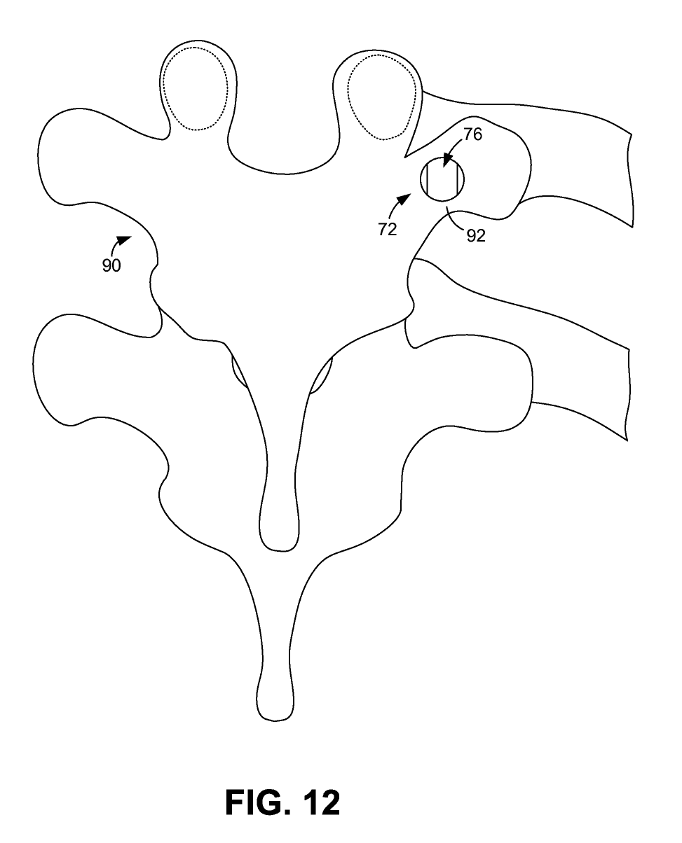

FIGS. 11 and 12 illustrate the attachment element 70 of FIGS. 8 and 9 attached to a vertebra 90. As is shown in those figures, the shaft 74 of the attachment element 70 is passed through a transverse process 92 and is held in place by passing the retainer element 80 through the opening 78 at the distal end of the shaft.

FIG. 10 illustrates a fourth embodiment of a rod-receiving spinal fusion attachment element 100 that is similar in many ways to the attachment element 70 shown in FIGS. 8 and 9. Accordingly, the attachment element 100 comprises a rod attachment head 102 having a passage 104 and a shaft 106 that extends outward from the head. At the distal end of the shaft 106, however, are barbs or tines 108 that are adapted to anchor the attachment element 100 in place within the transverse process.

Although a particular configuration for the barbs/tines is shown in FIG. 10, many other configurations are possible. Moreover, the barbs/tines can be contained within the shaft 106 during placement of the attachment element 100 and then deployed once the desired position has been reached. In some cases, the barbs/tines can deploy in similar manner to that of drywall anchors. The particular configuration that is used is not important as long as the barb/tines secure the attachment element 100 in place and prevent it from being unintentionally removed from the transverse process.

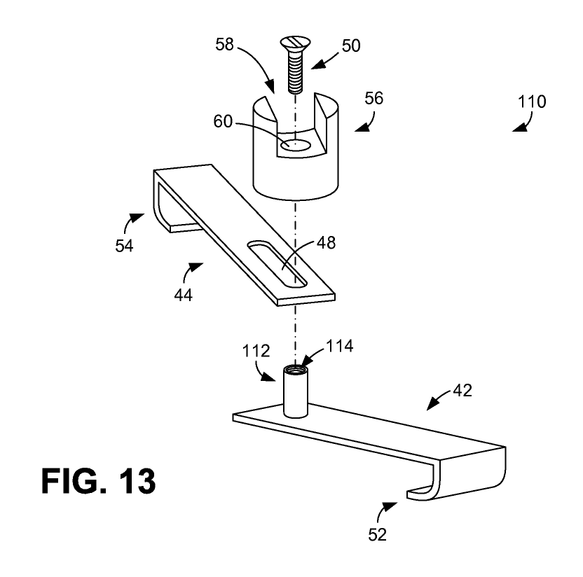

FIG. 13 illustrates a fourth embodiment of a rod-receiving spinal fusion attachment element 110, which is similar in many ways to the attachment element 40 shown in FIGS. 3 and 4. In the embodiment of FIG. 13, however, the first or lower arm 42 comprises a threaded element 112 into which the fastener 50 can threaded. In the illustrated embodiment, the threaded element 112 comprises a cylindrical stud that extends upwardly from an outer surface of the lower arm 42. The stud comprises a threaded opening 114 into which the fastener 50 can be threaded. As is indicated in FIG. 13, the stud can pass through the opening 48 in the upper arm 44 and into the opening 60 formed in the rod attachment head 56. By tightly threading the fastener 50 into the 114 of the stud, the head 56, upper arm 44, and lower arm 42 can be secured together in a desired orientation. In some embodiments, the head 56 can pivot about the top of the 102.

Claimed are:

1. An implantable spinal fusion attachment element comprising:
a rod attachment head having a passage that is adapted to receive a spinal alignment rod; and
means for securely attaching the attachment element to a vertebra without penetrating the pedicle of the vertebra.

2. The spinal fusion attachment element of claim 1, wherein the head is a multiaxial tulip head.

3. The spinal fusion attachment element of claim 1, wherein the means for securely attaching comprise first and second arms adapted to grip the vertebral body.

4. The spinal fusion attachment element of claim 3, wherein the first and second arms each comprise a distal end that includes a hook that is adapted to grip the vertebral body.

5. The spinal fusion attachment element of claim 3, wherein the first and second arms each comprise a proximal end that includes an opening adapted to receive a fastener that fastens the arms together.

6. The spinal fusion attachment element of claim 5, wherein the opening of the second arm is an elongated slot.

7. The spinal fusion attachment element of claim 1, wherein the means for securely attaching comprise a shaft that extends from the head and a retainer element associated with the shaft.

8. The spinal fusion attachment element of claim 7, wherein the retainer element comprises a small rod that passes through an opening formed in the distal end of the shaft.

9. The spinal fusion attachment element of claim 7, wherein the retainer element comprises at least one barb or tine that extends out from the shaft.

10. The spinal fusion attachment element of claim 9, wherein the barb or tine can be deployed to extend out from the shaft.

11. An implantable spinal fusion system comprising:
spinal fusion attachment elements, at least one of the attachment elements including a rod attachment head having a passage that is adapted to receive a spinal alignment rod and means for securely attaching the attachment element to a vertebra without penetrating the pedicle of the vertebra; and
a spinal alignment rod that connects to the heads of the attachment elements.

12. The spinal fusion system of claim 11, wherein the head is a multiaxial tulip head.

13. The spinal fusion system of claim 11, wherein the means for securely attaching comprise first and second arms adapted to grip the vertebral body.

14. The spinal fusion system of claim 13, wherein the first and second arms each comprise a proximal end that includes an opening adapted to receive a fastener that fastens the arms together and a distal end that includes a hook that is adapted to grip the vertebral body.

15. The spinal fusion system of claim 1, wherein the means for securely attaching comprise a shaft that extends from the head and a retainer element associated with the shaft.

16. The spinal fusion system of claim 15, wherein the retainer element comprises a small rod that passes through an opening formed in the distal end of the shaft.

17. The spinal fusion system of claim 15, wherein the retainer element comprises at least one barb or tine that extends out from the shaft.

18. A method for fusing vertebrae in a desired configuration, the method comprising:
attaching spinal fusion attachment elements to the vertebrae, at least one of the attachment elements being securely attached to a vertebra without penetrating the pedicle of the vertebra; and
connecting the attachment elements with an alignment rod.

19. The spinal fusion system of claim 18, wherein attaching comprises gripping the vertebral body with first and second arms of the attachment element, the arms comprising a proximal end that includes an opening adapted to receive a fastener that fastens the arms together and a distal end that includes a hook that is adapted to grip the vertebral body.

20. The spinal fusion system of claim 18, wherein attaching comprises passing a shaft of the attachment element through a transverse process of the vertebra.
